(19) United States
(12) Patent Application Publication
(10) Pub. No.: US 2001/0009673 A1
(43) Pub. Date: Jul. 26, 2001

(54) TRANSDERMAL THERAPEUTIC SYSTEMS

(76) Inventors: RALPH LIPP, BERLIN (DE); CLEMENS GUNTHER, BERLIN (DE)

Correspondence Address: MILLEN WHITE ZELANO & BRANIGAN, ARLINGTON COURTHOUSE PLAZA I, 2200 CLARENDON BOULEVARD, SUITE 1400, ARLINGTON, VA, 22201

(21) Appl. No.: 09/214,601

(22) PCT Filed: Jul. 11, 1997
(86) PCT No.: PCT/DE97/01517

§ 371 Date: None

(30) Foreign Application Priority Data
Jul. 11, 1996 (DE) 196 29 468.1

(51) Int. Cl. A61K 9/00

(52) U.S. Cl. 424/400

(57) ABSTRACT

Transderml therapeutic systems are disclosed for the controlled administration of second and third generation gestagenes, and possibly of oestrogens, at least partially dissolved in a matrix. The disclosed systems are characterised in that their matrix contains a copolymer of one or several esters or amides which contain vinyl groups and have the general formula (I), as well as an N-vinyl lactam of general formula (II). In the general formula (I), X symbolises a -COO- group, a -CONH- group or an -OCO- group; R₁ stands for a hydrogen atom or an alkyl group with maximum 4 carbon atoms; and R₂ stands for a hydrogen atom or an alkyl group with maximum 12 carbon atoms. In the general formula (II), n is a digit from 3 to 5.

The invention relates to transdermal therapeutic systems for the controlled release of gestagens of the second and third generations, which are at least partially dissolved in a matrix and are optionally of estrogens, which are characterized in that their matrix is a copolymer of one or more vinyl group-containing esters or amides of general formula (I),

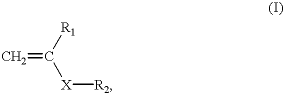

in which

X symbolizes a —COO group, a —CONH group or an —OCO group,

R₁ means a hydrogen atom or an alkyl group with a maximum of 4 carbon atoms, and

R₂ represents a hydrogen atom or an alkyl group with a maximum of 12 carbon atoms, and an N-vinyllactam of general formula II,

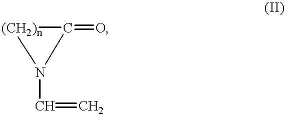

in which

n means a number from 3 to 5, whereby the steroidal active ingredients that are dissolved in the matrix essentially do not crystallize out over a prolonged period.

The invention relates in particular to those transdermal therapeutic systems whose matrix is a copolymer, which per mol of N-vinyllactone of general formula II contains 0.5 to 2.5 mol and preferably 0.75 to 1.5 mol of one or more vinyl group-containing esters or amides of general formula I.

Transdermal therapeutic systems (TDS) are, as is generally known, plasters that are made of many layers, which are attached to the skin and which continuously release the active ingredient percutaneously over a prolonged period. Transdermal therapeutic systems essentially consist of a cover film that is impermeable to water, penetration enhancers and active ingredients, a matrix that comprises the skin contact adhesive, penetration enhancers and pharmaceutical substance, and a detachable protective film.

High concentrations of dissolved active ingredient in the matrix of transdermal therapeutic systems generally make possible a high flow of active ingredients through the skin. In particular, there have been frequent reports recently of so-called supersaturated systems, which make possible the desired high transdermal flow of pharmaceutical substances (K. H. Ziller et al., Pharm. Ind. 52, (1990), 1017 ff).

A problem of such supersaturated solutions is the insufficient storage stability. Since sparingly soluble, easily crystallizing compounds are involved in the incorporated active ingredients, as is the case with gestagens and estrogens, crystallization processes must be expected during storage. This tendency toward crystal formation or toward crystal growth respectively is known, for example, in the case of suspensions and supersaturated solutions of steroid hormones (M. Kunert-Brandstätter et al., Sci. Pharm., 35 (1967), 287 ff). This phenomenon also applies to supersaturated solutions of sparingly soluble substances in acrylate adhesive-enhancer mixtures.

Because of the crystallization process, the portion is shifted from dissolved active ingredient to crystallized active ingredient. In this case, the value in question may optionally drop even below the saturation concentration of the active ingredient in the system (Jian-wei Yu et al., Drug Development and Industrial Pharmacy 17, (1991), 1883 ff). In addition, crystal growth leads to the reduction of the crystal surfaces, by which the rate of solution is reduced during the administration.

Japanese Patent Applications JP-A 02,196,714—(ref. C.A. 113, 1990) and JP-A 04,342,532 described transdermal therapeutic systems that contain norethisterone as a gestagen of the so-called first generation or estradiol as active ingredients in a matrix of a copolymer that consists of acrylic acid-2-ethylhexyl ester and N-vinylpyrrolidone.

The object of this invention was to make available transdermal therapeutic systems that contain gestagens of the so-called second and third generations optionally in combination with estrogen, which are characterized in that the steroidal active ingredients essentially do not crystallize out.

This object was achieved by this invention.

Gestagens of the so-called second and third generations are, for example, levonorgestrel, gestodene, desogestrel, 3-keto-desogestrel, norgestimate, dienogest, dihydrospirenone and cyproterone as well as its acetate.

Suitable estrogens according to this invention are both natural estrogens, such as, for example, estradiol, as well as its esters, such as, for example, estradiol ester, and synthetic estrogens, such as ethinylestradiol and especially 14α,17α -ethanoestra-1,3,5(10)-triene-3,17β-diol (WO-88/01275) and 14α,17α-ethanoestra-1,3,5(10)-triene-3,16α,17β-triol (WO-91/08219).

The copolymers that are required as a matrix are available commercially, such as, for example, the copolymer that is contained in the “TSR Adhesive Solution” of the Japanese Sekisui Company.

The transdermal systems according to the invention can optionally in addition contain 1 to 35%, preferably 5 to 20% of a water-soluble polymer of vinylpyrrolidone, such as, e.g., polyvinylpyrrolidone (e.g., Kollidon 12PF, Kollidon 17PF, Kollidon 25, Kollidon 30 of the BASF Company, Ludwigshafen, Germany or a water-soluble copolymer of vinylpyrrolidone and vinyl acetate (e.g., Kollidon VA 64, BASF).

In addition, however, they can also be produced in a way that is known in the art by, for example, a solution or emulsion of the vinyl group-containing esters or amides of general formula I and the N-vinyllactams of general formula II being polymerized in the presence of radical formers such as peroxides (such as benzoyl peroxide or lauryl peroxide), as well as optionally cross-linking agents, adjustment substances and telogens (such as hexamethyldiisocyanate, diphenylmethanediisocyanate, hexamethylene glycol and trimethylpropanol-triacrylate) (Houben-Weyl: “Methoden der organischen Chemie [Methods of Organic Chemistry],” Georg Thieme Verlag, DE-Stuttgart, 4th Edition, Volume XIV/1, p. 100 ff).

Suitable vinyl group-containing esters and amides of general formula I are, for example:

a) Vinyl esters, such as vinyl acetate, vinyl propionate, vinyl pivalate or vinyl-2-ethylhexanoate (Ullmann's Encyclopedia of Industrial Chemistry, 5th Edition, Volume A 22, 1993, 1 ff),

b) Methacrylic acid ester, such as methacrylic acid methyl ester, methacrylic acid-tert-butyl ester, methacrylic acid allyl ester and methacrylic acid-2-ethylhexyl ester (Ullmann's Encyclopedia of Industrial Chemistry, 5th Edition, Volume A 21, 1992, 473 ff) and especially

c) Acrylic acid and its esters and amides, such as acrylic acid amide, acrylic acid methyl ester, acrylic acid ethyl ester, acrylic acid-2-hydroxyethylester, acrylic acid allyl ester and acrylic acid-2-ethylhexylester (Ullmann's Encyclopedia of Industrial Chemistry, 5th Edition, Volume A 21, 1992, 157 ff).

Suitable vinyllactams of general formula II are N-vinyl piperidone, N-vinyl caprolactam and especially N-vinylpyrrolidone (Ullmann's Encyclopedia of Industrial Chemistry, 5th Edition, Volume A 21, 752 ff).

The production of the transdermal therapeutic systems according to the invention itself can also be implemented under the conditions that are well known to one skilled in the art (Yie W. Chien: “Transdermal Controlled Systemic Medications,” Marcel Dekker Inc., New York and Basel, 1987, Dr. Richard Baker: “Analysis of Transdermal Drug Delivery Patents 1934 to 1984” and “Analysis of Recent Transdermal Delivery Patents, 1984-1986 and Enhancers”; see also, for example, WO 90/04398, WO 93/08795 and WO 95/22322).

Thus, for example, such a transdermal therapeutic system can be produced that consists of

a) an impermeable cover layer, one to three matrix layer(s) that adhere(s) to the cover layer and that contain(s) the active ingredient(s) and optionally penetration-enhancing agents and other adjuvants, whereby said matrix layer(s) consist(s) of a copolymer that is self-adhesive or is (are) covered or surrounded by a skin contact adhesive that optionally contains penetration-enhancing agents of one or more vinyl group-containing esters or amides of general formula I

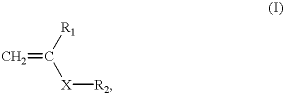

in which

X symbolizes a —COO group, a —CONH group or an —OCO group,

R₁ means a hydrogen atom or an alkyl group with a maximum of 4 carbon atoms, and

R₂ represents a hydrogen atom or an alkyl group with a maximum of 12 carbon atoms, and an N-vinyllactam of general formula II,

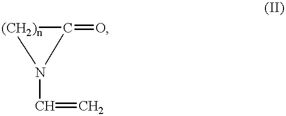

in which

n means a number from 3 to 5, and a removable protective layer, or of

b) a cover that is provided with a contact adhesive that optionally contains penetration-enhancing agents; one or two matrix layer(s) that is (are) fastened to the contact adhesive by a cover that allows a contact adhesive border to be uncovered, whereby said matrix layer(s) contain(s) the active ingredient(s) and optionally penetration-enhancing agents and other additives and consist(s) of a copolymer of a vinyl group-containing ester and an amide of general formula I

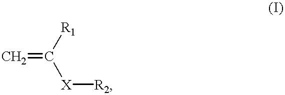

in which

X symbolizes a —COO group, a —CONH group or an —OCO group,

R₁ means a hydrogen atom or an alkyl group with a maximum of 4 carbon atoms, and

R₂ means a hydrogen atom or an alkyl group with a maximum of 12 carbon atoms, and an N-vinyllactam of general formula II,

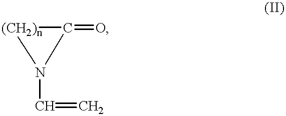

in which

n means a number from 3 to 5, and a removable protective layer.

A transdermal therapeutic system according to variant a) represents a simple matrix system. It can be, for example, round, oval or rectangular in shape and can be produced, for example, as follows:

A solution of up to 25% by weight of active ingredient or active ingredient mixture and penetration-enhancing agents, 30 to 70% by weight of the copolymer, made up to 100% by weight with a suitable volatile solvent is painted on a flat, impermeable cover layer. After drying, a second and optionally later even a third layer that optionally contains active ingredient, penetration-enhancing agents and adhesive can be applied to this layer and dried. Then, the matrix system is provided with a removable protective layer.

If a copolymer is used as a matrix system, which, after the system is dried, does not adhere or adheres only inadequately to the skin, the system can additionally be covered or surrounded with a skin contact adhesive before the removable protective layer is applied.

Suitable volatile solvents are, for example, lower alcohols, ketones or esters of lower carboxylic acids such as ethanol, isopropanol, acetone or ethyl acetate, polar ethers such as tetrahydrofuran, lower hydrocarbons, such as cyclohexane, benzene or toluene or else lower halogenated hydrocarbons, such as dichloromethane or tetrachloroethane. It goes without saying that mixtures of these solvents are also suitable.

Suitable penetration-enhancing agents are, for example, monovalent or multivalent alcohols, such as ethanol, 1,2-propanol, or benzyl alcohol, fatty alcohols or fatty acids with 8 to 18 carbon atoms, such as lauryl alcohol, cetyl alcohol, stearic acid or oleic acid, or fatty acid esters and dicarboxylic acid diesters with up to 24 carbon atoms. Fatty acid esters, which are suitable as penetration enhancers, are, for example, those of acetic acid, caproic acid, lauric acid, myristic acid and palmitic acid, such as, for example, methyl ester, ethyl ester, isopropyl ester, tert-butyl ester or monoglyceric acid ester. Especially preferred esters are those of myristic acid or oleic acid, such as their methyl esters or especially their isopropyl esters. Suitable dicarboxylic acid esters are, for example, diisopropyl adipate, diisobutyl adipate and diisopropyl sebacate.

Other penetration-enhancing agents are phosphatide derivatives, such as lecithin, terpenes, amides, ketones, urea and its derivatives or ethers, such as, for example, dimethyl isosorbide. Mixtures of these penetration-enhancing agents are, of course, also suitable.

As protective layers, all films are suitable that are usually used in transdermal therapeutic systems. Such films are, for example, siliconized or fluoropolymer-coated.

As cover layers, in these systems, for example, 10 to 100 μm thick films of polyethylene or polyesters can be used either pigmented or metallized. The pharmaceutical agent layer that is applied to this preferably has a thickness of 20 to 500 μm. The release of active ingredients is preferably carried out over a surface area of 5 to 100 cm².

In the case of multilayer matrix systems, the gestagen and optionally penetration enhancers can be introduced into, for example, the matrix that is applied to the impermeable cover layer, while the underlying layer or layers contain(s) the estrogens and optionally also penetration enhancers. In addition, it is also possible, however, to arrange several active ingredient-containing matrix layers beside one another in such a transdermal system.

A transdermal therapeutic system according to variant b) can also be, for example, round, oval or rectangular, and can be produced as follows:

A cover is coated with a skin contact adhesive. Then, in each case, one to three areas that are provided with a cover and are produced according to variant a) and that contain the active ingredient(s) and optionally penetration-enhancing agents are glued to this cover in such a way that the cover has an adequate edge for attaching to the skin and also adequate gaps in several areas, and it is provided with a removable protective layer. The materials that are used in these matrix systems can be the same as those that are used in variant a).

The transdermal therapeutic systems according to the invention are distinguished from those systems that are known from the prior art in that even at a very high concentration of gestagens of the second and third generations optionally in combination with an estrogen in the matrix over a prolonged period, essentially no active ingredient crystals are formed.

It was already mentioned that the agents according to the invention have an excellent shelf life. Thus, for example, transdermal therapeutic systems, which contain 5% gestodene, 2% levonorgestrel, mixtures of 2% levonorgestrel and 2% estradiol or of 4% gestodene and 4% estradiol in a skin contact adhesive of the Sekisui Company (that consists of a copolymer of 35% vinylpyrrolidone and 65% 2-ethylhexylacrylate), are crystal-free in the microscopic image even after being stored for 10 months at 25° C., while the corresponding systems contain crystals in a polymer of pure 2-ethylhexylacrylate even after being stored for three months at 25° C.

Essentially crystal-free according to this invention is defined as the transdermal therapeutic systems according to the invention being crystal-free in the microscopic image at a 2% gestagen concentration over a storage period of 25 months at 25° C.

The concentration of the active ingredient or the active ingredients in the transdermal therapeutic systems according to the invention depends, of course, on the type of active ingredient that is used and in the case of gestagens and estrogens is usually 0.1 to 25% by weight, whereby 0.5 to 5% by weight is preferred. Especially preferred is a concentration of 0.5 to 2.5% levonorgestrel and 1 to 5% estradiol.

The embodiments below are used only for a more detailed explanation of the transdermal therapeutic systems according to the invention and are in no way limiting.

### EXAMPLE 1

0.5 g of levonorgestrel is suspended in 5 g of isopropanol and added to 68.1 g of a 36% solution of a copolymer of vinylpyrrolidone and 2-ethylhexylacrylate in ethyl acetate (TRS-Adhesive Solution of the Sekisui Company, Osaka, Japan) after 30 minutes of stirring. It is made up to 95 g with isopropanol and stirred until all levonorgestrel is dissolved. Optionally, the respective feedstock mixture is heated to 35° C. or treated with ultrasound. By means of a doctor blade, the crystal-free solution is painted onto a release liner (e.g., polyester film that is siliconized on one side, produced by the Bertek Company, Vermont, U.S.A.) in such a way that the dry coating weight of 100 g/m²is produced.

Subsequently, the volatile solvents are removed by 20 minutes of drying at 70° C. and then a cover film (=backing, e.g., saran-hytel-colaminate, produced by the Bertek Company, Vermont, U.S.A.) is laminated on the adhesive matrix. Transdermal systems of 20 cm² are punched out from the three-layer product that is thus obtained and are individually packaged.

### EXAMPLE 2

A transdermal therapeutic system is produced under the conditions of Example 1, but with additional use of 1.0 g of estradiol, whereby the portion of TSR adhesive solution that corresponds to 66.7 g is reduced.

### EXAMPLE 3

A transdermal therapeutic system is produced under the conditions of Example 1, but with use of 2.0 g of gestodene and 63.9 g of TSR adhesive solution.

### EXAMPLE 4

A transdermal therapeutic system is produced under the conditions of Example 1, but with use of 2.0 g of gestodene, 2.0 g of estradiol, 5.0 g of lauryl alcohol and 58.3 g of TSR adhesive solution.

### EXAMPLE 5

100.0 g of acrylic acid-2-ethylhexylester, 32.5 g of N-vinylpyrrolidone and 15 mg of hexamethylene glycol-di-methacrylic acid ester are mixed with 23.4 g of ethyl acetate. Then, the solution, which contains 85% by weight of monomers, is heated to 60° C., and it is slowly mixed in portions in a nitrogen atmosphere with lauryl peroxide in ethyl acetate and polymerized for 32 hours.

The polymer solution in ethyl acetate that is thus obtained can be used in the same way as the commercially available TRS-adhesive solution in Examples 1 to 4 for the production of transdermal therapeutic systems.

### EXAMPLE 6

### Production of a TTS with 2% Levonorgestrel in the Matrix

242 mg of levonorgestrel is suspended in 5 g of isopropanol and 6.0 g of a 30% solution of a copolymer of vinylpyrrolidone and vinyl acetate (Kollidon VA 64 of the BASF Company, Ludwigshafen, Germany), and after 5 minutes of stirring and 30 minutes of ultrasonic acoustic irradiation, it is added to 28.64 g of a 36% solution of a copolymer of vinylpyrrolidone and 2-ethylhexylacrylate in ethyl acetate (TRS-adhesive solution of the Sekisui Company, Osaka, Japan). It is made up to 95 g with isopropanol and stirred until the feedstock is crystal-free. By means of a doctor blade, the crystal-free solution is painted onto a release liner (e.g., polyester film that is siliconized on one side, produced by the Bertek Company, Vermont, U.S.A.) in such a way that the dry coating weight of 100 g/m² is produced.

Subsequently, the volatile solvents are removed by 20 minutes of drying at 70° C. and then a cover film (=backing, e.g., saran-hytel-colaminate, produced by the Bertek Company, Vermont, U.S.A.) is laminated on the adhesive matrix. Transdermal systems of 20 cm² are punched out from the three-layer product that is thus obtained and are individually packaged.

For transdermal therapeutic systems with other concentrations of levonorgestrel or estradiol-hemihydrate, the quantities that are presented in the following table are used analogously to Example 6:

| | | | | | Weighed | |
|---|---|---|---|---|---|---|
| | | | | | portion | Weighed |
| | % LNG | | | | of 30% | portion |
| Ex- | in | % E2 in | Weighed | Weighed | Kollidon | of 36% |
| am- | the | the | portion | portion | VA 64 | Sekisui |
| ple | matrix | matrix | of LNG | of E2 | solution | solution |
| 7 | 2 | 4 | 240 mg | 484 mg | 6.10 g | 27.10 g |
| 8 | 1 | 2 | 122 mg | 242 mg | 6.19 g | 28.11 g |
| 9 | 0.5 | 1 | 61 mg | 122 mg | 6.00 g | 28.77 g |
| 10 | 2.5 | 5 | 302 mg | 608 mg | 6.09 g | 26.69 g |
| 11 | 2.5 | — | 301 mg | 0 | 5.94 g | 28.29 g |

### Determination of Percutaneous Resorption

The determination of the extent and speed of percutaneous resorption by the agent according to the invention can be carried out using in vitro testing.

The freshly-prepared complete skin of nude mice from which subutaneous fat is removed is clamped in a Franz diffusion cell, which as an acceptor medium contains 50% polyethylene glycol-(MW 400)solution. After administration of a matrix-TTS, the concentration time plot of the steroid in the acceptor medium is determined using suitable analysis. The matrix-TTS that contains levonorgestrel and estradiol that is cited in Table 1 was tested in this model.

TABLE 1
Steroid Concentration of the Tested Matrix-TTS

| Example | Levonorgestrel | Estradiol |
|---|---|---|
| 6 | 2% | — |
| 7 | 2% | 4% |
| 8 | 1% | 2% |
| 9 | 0.5% | 1% |

Table 2 below shows the results that are obtained in this test:

the percutaneous resorption of levonorgestrel increases as a function of the steroid concentration of TTS

the additional concentration of the levonorgestrel-TTS with estradiol does not affect the percutaneous resorption of levonorgestrel

TABLE 2
Average Percutaneous Levonorgestrel Flow [ng/cm²/h] In
Vitro after Administration of TTS on the Skin of Nude Mice

| Example | Levonorgestrel | Estradiol | |
|---|---|---|---|
| 7 | 2% | 4% | 237 |
| 8 | 1% | 2% | 125 |
| 9 | 0.5% | 1% | 50 |

The results show that the percutaneous levonorgestrel flow with the gents according to the invention relative to other TTS, which represent the present state of the art, can be improved, since the agent according to the invention allows a higher steroid concentration.

1. Transdermal therapeutic systems for the controlled release of gestagens of the second and third generations, which are at least partially dissolved in a matrix and are optionally of estrogens, characterized in that their matrix is a copolymer of one or more vinyl group-containing esters or amides of general formula (I), 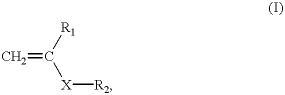
in which
X symbolizes a —COO group, a —CONH group or an —OCO group,
R₁ means a hydrogen atom or an alkyl group with a maximum of 4 carbon atoms, and
R₂ represents a hydrogen atom, or an alkyl group with a maximum of 12 carbon atoms, and an N-vinyllactam of general formula II, 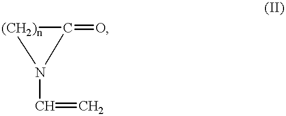
in which
n means a number from 3 to 5, whereby the steroidal active ingredients that are dissolved in the matrix essentially do not crystallize out over a prolonged period.

2. Transdermal therapeutic system according to claim 1, whose matrix is a copolymer, which per mol of N-vinyllactone of general formula II contains 0.5 to 2.5 mol of one or more vinyl group-containing esters or amides of general formula I.

3. Transdermal therapeutic system according to claim 1, whose matrix is a copolymer, which per mol of N-vinyllactone of general formula II contains 0.75 to 1.5 mol of one or more vinyl group-containing esters or amides of general formula I.
